(19) United States
(12) Patent Application Publication
(10) Pub. No.: US 2010/0312198 A1
(43) Pub. Date: Dec. 9, 2010

(54) INSERT FOR TREATING HUMAN OR ANIMAL BODY CAVITIES, IN PARTICULAR EAR CAVITIES

(76) Inventor: Gianmaria Guidi, Brescia (IT)

Correspondence Address: CASTELLANO PLLC, P.O. Box 1555, Great Falls, VA, 22066, US

(21) Appl. No.: 12/445,965

(22) PCT Filed: Oct. 19, 2007
(86) PCT No.: PCT/IT07/00732

§ 371 Date: Aug. 26, 2010

(30) Foreign Application Priority Data
Oct. 20, 2006 (IT) PD2006A000389

(51) Int. Cl. A61F 11/00; A61M 31/00

(52) U.S. Cl. 604/257; 606/162; 604/285

(57) ABSTRACT

An insert (1) for treating human or animal body cavities, in particular ear cavities, comprises an operating element (2) which is located at one end of the insert intended to be introduced into the cavity and to come into contact with the internal walls of the latter, and is expandable from a radially compressed configuration, in which it may be introduced into the cavity without substantial interference with the internal walls, to a radially expanded configuration having a substantially larger cross-section relative to the compressed configuration, in which radially expanded configuration the operating element (2) is pressed against the internal walls of the cavity.

### FIELD OF THE INVENTION

The present invention relates to an insert for treating human or animal body cavities, in particular ear cavities, according to the features set out in the precharacterising clause of the main claim.

### BACKGROUND OF THE INVENTION

The invention is preferably, although not exclusively, used in the technical field of manufacturing personal hygiene products, in particular products for cleaning the ear cavities.

Inserts in the form of a stick provided at the axially opposite ends thereof with a covering of cotton wool are commonly known and used, being intended to be introduced manually into the ear cavities and brought into contact with the internal walls thereof, in such a manner as to remove any grease and dirt present there.

Such known products do, however, exhibit some disadvantages, one of which is the possibility of compressing any dirt to be removed against the tympanic membrane instead of extracting it.

Another limiting factor is that the known sticks are not well suited to removing any excess water present in the ear canal.

A further disadvantage is that the stick's cleaning capacity is substantially proportional to the force with which the user applies the end of the stick against the internal walls of the ear cavities, which amounts to a potential hazard for the delicate internal parts of the ear, such as the tympanic membrane.

Furthermore, such sticks have a tendency to shed filaments of cotton within the ear cavity, so bringing about an increase in the production of earwax which then has to be removed.

Another known cleaning method involves using detergent liquids which are introduced into the ear cavities with suitable dispensers and are capable of at least partially dissolving the earwax. However, such liquids may act as a vector capable of transporting pieces of earwax into small cavities which it cannot otherwise reach, for example beyond the eardrum, with a consequent risk of inflammation and infection.

### DESCRIPTION OF THE INVENTION

The problem underlying the present invention is that of providing an insert for treating human and animal cavities, in particular ear cavities, which is structurally and functionally designed to overcome the limitations described above with reference to the cited prior art.

Said problem has been solved by the present invention by means of an insert produced according to the claims stated below.

### BRIEF DESCRIPTION OF THE DRAWINGS

The features and advantages of the present invention will become clear from the following detailed description of some of the preferred embodiments thereof, which are given purely by way of non-limiting example with reference to the appended drawings in which:

FIG. 1 is a schematic view in partial longitudinal section of a first example of an insert produced according to the present invention, in a radially compressed configuration,

FIG. 2 is a view in partial longitudinal section of the insert of FIG. 1 in a radially expanded configuration,

FIGS. 2a to 2c are schematic, partially sectional schematic views of the insert of FIG. 1 in successive operating positions,

FIG. 3 is an exploded view in longitudinal section of the insert of FIG. 2,

FIG. 4 is a view in partial longitudinal section of a first embodiment of the insert of FIG. 1,

FIG. 5 is a cross-sectional view according to line V-V of the insert of FIG. 4,

FIG. 6 is a schematic view showing use of the insert of FIG. 4,

FIG. 7 is a schematic view in partial longitudinal section of a second embodiment of the insert of FIG. 1,

FIGS. 8 and 9 are views in partial longitudinal section of a variant embodiment of the insert of FIG. 1, respectively in radially compressed and radially expanded configuration,

FIGS. 10 and 11 are views in partial longitudinal section of another variant embodiment of a component of the insert of FIG. 1, respectively in radially compressed and radially expanded configuration,

FIGS. 12 to 14 are views in partial longitudinal section of a second example of an insert produced according to the present invention, in successive operating positions,

FIGS. 15 to 16 are views in partial longitudinal section of a third example of an insert produced according to the present invention, in successive operating positions,

FIGS. 17 to 18 are views in partial longitudinal section of a fourth example of an insert produced according to the present invention, in successive operating positions,

FIGS. 19a, 19b, and 19c are partially sectional schematic views of another variant embodiment of the insert of FIG. 1 in successive operating positions,

FIG. 20 is an axonometric view of another variant embodiment of the insert of FIG. 1,

FIG. 21 is an exploded view of the variant embodiment of FIG. 20.

### PREFERRED EMBODIMENTS OF THE INVENTION

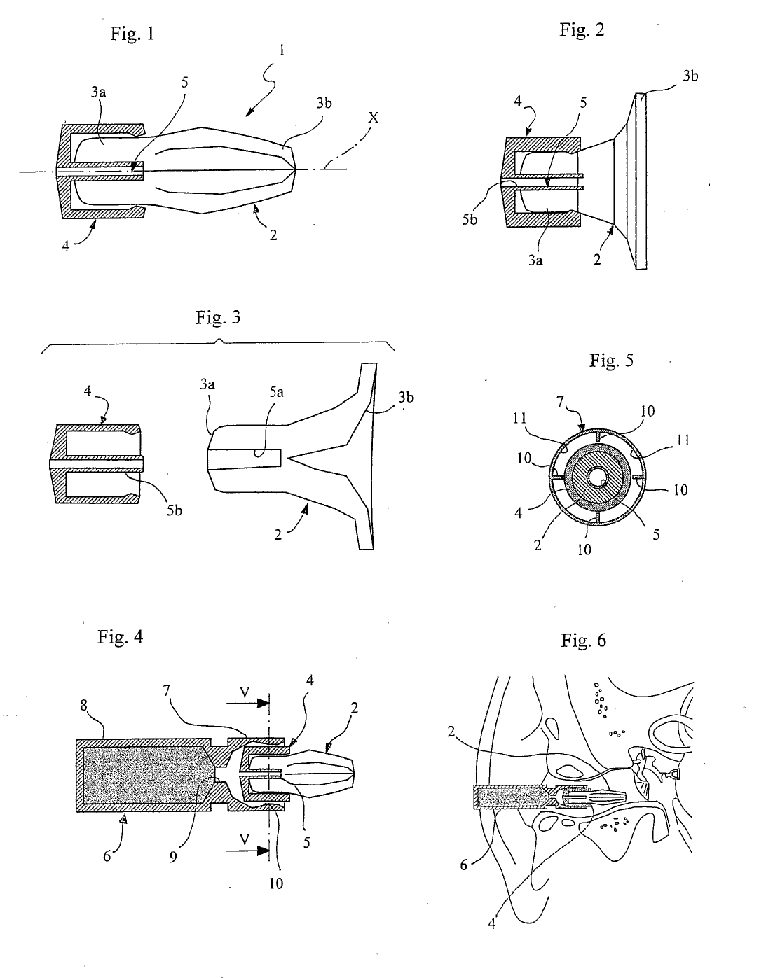

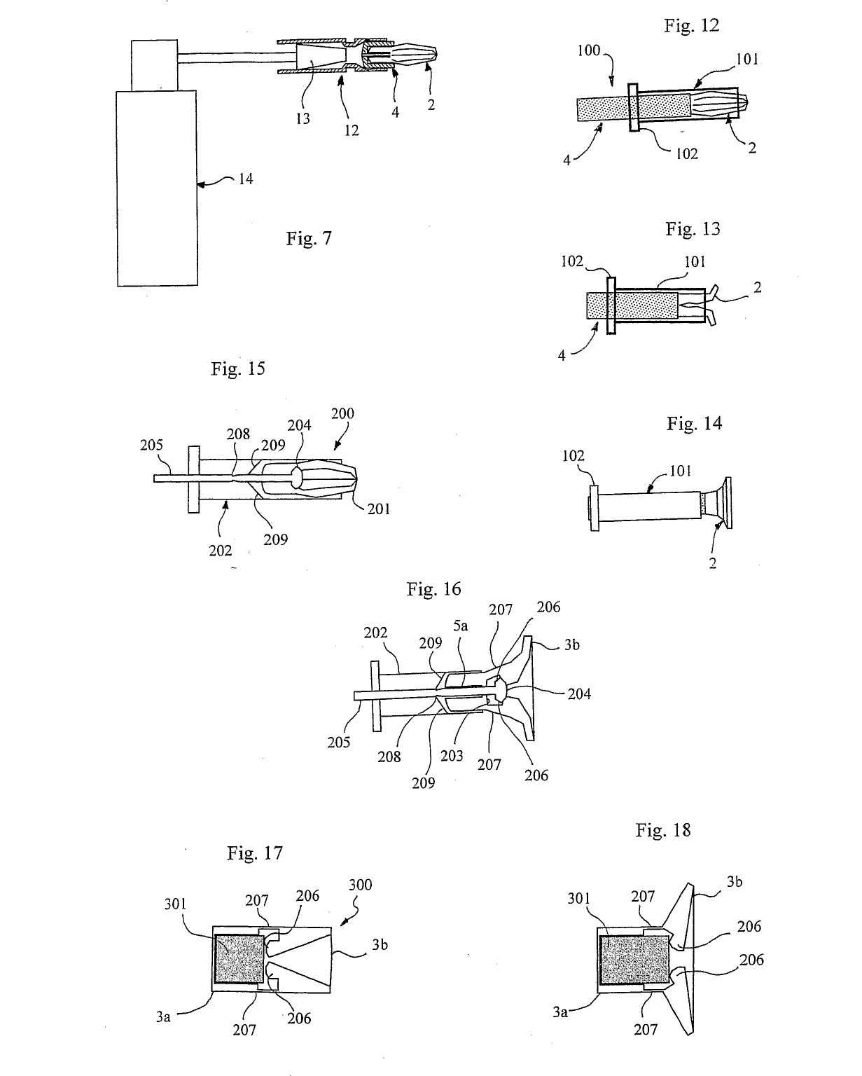

With reference initially being made to FIGS. 1 to 6, 1 denotes overall an insert for treating human and animal body cavities produced according to the present invention.

The insert 1, as shown in FIG. 6, is in particular designed for use in cleaning the ear cavities in human beings, but the inventive concept on which the present invention is based may be used effectively in similar applications for cleaning or medicating body cavities both in human beings and in animals.

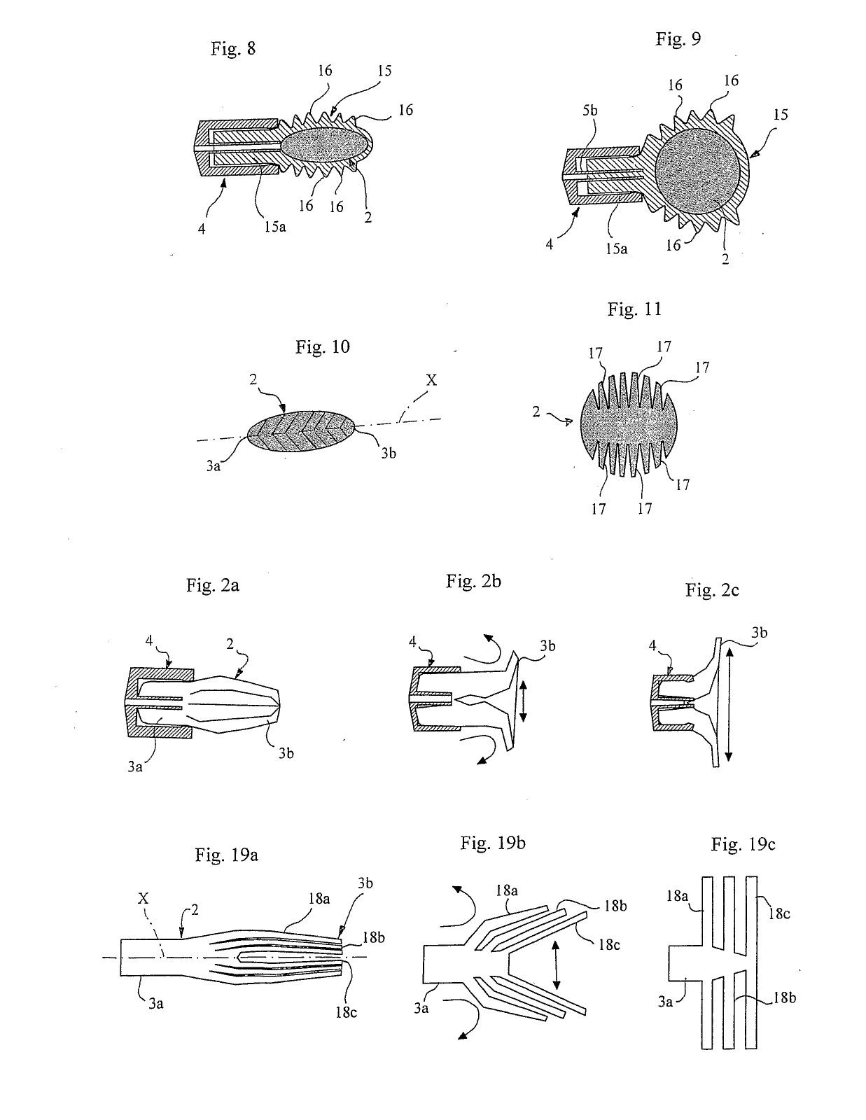

The insert 1 comprises an operating element 2 extending along a longitudinal axis X which is capable of changing, by the methods described in greater detail below, from a radially compressed configuration, which is cylindrical overall (FIG. 1), to a radially expanded configuration (FIG. 2).

A first end 3a of the operating element 2 is surrounded and held by a substantially rigid collar member 4, made for example from a plastics or cellulosic material. The opposite free end 3b of the operating element 2, in contrast, is free to expand radially, at the same time turning towards the first end 3a (turning back), such that the insert assumes overall a more or less conical, preferably bell-like, shape.

In greater detail, the free end 3b may be produced in such a manner as to have, in its radially expanded configuration, a single continuous wall, of circular cross-section, or, in a similarly preferred manner, a plurality of tabs which are separate from one another, in the manner of flower petals. It is understood that, in the present context, the phrase “bell-like shape” encompasses both of the above-stated options.

In this first example of embodiment of the invention, the operating element 2 is made of a hydrophilic material which is expandable by absorption of a liquid, such as for example compressed plant cellulose (similar to that used for sponges), or compressed hydrophilic cotton, and is comprises an axial passage 5a, open at the first end 3a, into which is introduced a tubular attachment 5b extending coaxially from the collar member 4, which attachment is open at both its axial ends. In this manner, an internal tubular duct 5 is defined in the insert 1, which duct is capable of passing a liquid from outside the insert, through the collar member 4, to the operating element 2.

The latter may conveniently be provided at the free end 3b with attachments which extend radially inwards, one against the other, such that, once they have expanded, they repel one another promoting the radial expansion of the operating element 2, and in particular the expansion and turning back movement of the free end 3b, until the final, bell-like shape is obtained.

In a first embodiment shown in FIG. 4, there may be engaged on the collar member 4, from the axially opposite side from the operating element 2, a container 6 containing a liquid to be introduced towards the operating element 2. The container 6, which may be a vial made of a soft plastics material, may conveniently be filled with a liquid comprising a detergent agent capable of promoting the detachment and removal of the dirt and grease which may be present in the ear canal. Alternatively or in addition, the liquid may comprise a medicinal agent, which is likewise to be introduced into the ear canal by means of the insert 1.

The container 6 preferably comprises a mouth 7 and a tank for holding the liquid 8 which is separated from the mouth 7 by a narrowing 9.

The mouth 7 is of a cylindrical shape with a circular cross-section and is engaged externally on the collar member 4. The mouth and the collar member are held together by longitudinal tongues 10 which extend radially from the mouth towards the collar member. In this manner, between adjacent tongues, respective circumferential openings 11 are defined, through which liquid may pass onto the outside of the operating element.

The insert 1 is produced and sold in its radially compressed configuration and it is introduced manually inside the ear cavity in this configuration.

Thanks to the small radial dimensions of the operating element, amounting to approx. 4-5 mm in diameter, the insert 1 is inserted without substantial interference with the internal walls of the ear canal (see FIG. 6).

When the insert has reached the correct position, it is possible by exerting pressure on the deformable container 6 to transfer the liquid contained therein both inside the operating element 2 through the tubular duct 5 and onto the outside of the operating element, directly in contact with the internal walls of the ear canal, through the circumferential openings 10.

The dispensed liquid, apart from optionally acting on the ear canal to dissolve the dirt, is absorbed by the operating element which changes into its radially expanded configuration by expansion and turning back of its free end 3b which, opening out like the petals of a flower, causes the insert 1 to assume a bell-like shape (see FIGS. 2a, 2b, 2c). It will be noted that, in its expanded configuration, the insert 1 has a cross-section (12-15 mm in diameter) which is appreciably larger than the initial cross-section (at least 3 times larger), and how this specific shape is particularly effective and safe because as a result the dirt is thrust towards the outside of the ear cavity, facilitating the extraction thereof without damaging any parts further within the ear cavity, in particular the eardrum.

It will be noted that, thanks to the features of the operating element 2, the insert 1 is conveniently capable of absorbing, by expansion, any excess liquid present in the ear cavity, even in the absence of the optional dispensing of liquid, so drying said cavity. In this application, the insert 1 may be used without the container 6.

Apart from the possibility of using liquid containers specifically sized to be engaged on the insert 1, as shown above, provision is also made to engage an adapter sleeve 12 onto the collar member 4, so making it possible to attach a spout 13 of existing, commercially available generic liquid containers 14 (see FIGS. 7, 20 and 21).

The mouth of the adapter sleeve 12 is preferably similar to the previously described mouth 7 of the container 6, and the opposite end of said sleeve 12 is open to permit introduction of the spout 13.

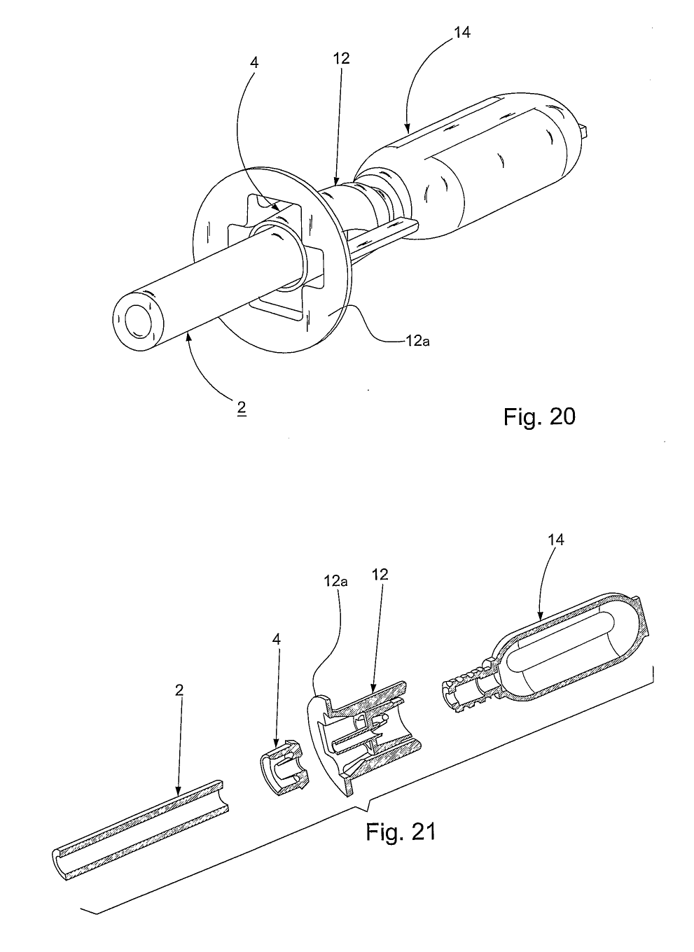

According to the variant embodiment of FIGS. 20 and 21, the adapter sleeve 12 comprises an annular projection 12a which extends radially towards the outside of the adapter sleeve 12 and is located in the vicinity of the engagement zone between the collar member 4 and the adapter sleeve 12. The annular projection 12a is capable of interfering with the outer ear in order to limit the distance the insert 1 is introduced into the ear cavity. Contact is consequently avoided between the insert 1 and the eardrum, preventing any damage to the eardrum itself.

According to another variant embodiment of the invention (not shown), the mouth 7 of the container 6 comprises an annular projection which is structurally and functionally similar to the annular projection 12a.

Provision is similarly made for the liquid to be introduced directly inside the operating element 2 through the tubular duct 5, for example by means of medicine dropper.

Apart from the preferred bell-like shape, it is also possible for the operating element 2 to be differently shaped, for example in an ellipsoidal or spherical shape, and for the internal passage 5a therein to be open to the outside through appropriate lateral ducts.

Another example of a preferred shape of the operating element 2 is shown in FIGS. 19a to 19c. According to this embodiment, the free end 3b is formed by a plurality of concentric crowns 18a, 18b and 18c arranged in close succession one after another along the longitudinal axis X.

The crowns 18a, b, c are separate and independent of one another and are capable of changing over from a radially compressed configuration, in which are they extend parallel to the axis X, coaxially closed around one another (FIG. 19a), to a radially expanded configuration in which they extend parallel to a plane almost perpendicular to the axis X (FIG. 19c).

In one variant embodiment of the invention, shown in FIGS. 8 and 9, the operating element 2 is covered externally by an elastic stocking 15, of the sterile gauze type, provided with raised stitching 16 to increase its dirt-removing capacity. In this case, the collar member 4 is preferably attached to a tail 15a of the elastic stocking 15, without directly affecting the operating element 2, and may be removed by being pulled out therefrom.

This embodiment is of particular benefit when the insert 1, alternatively or in addition to cleaning, is used as a member for medicating the ear cavity. A liquid containing a medicinal agent, such as a disinfectant, antibiotic or wound healing agent, may, for example, be introduced into the ear cavity by means of the insert 1, making use of the internal tubular duct 5. In this way, the operating element 2 is expanded against the internal walls of the ear cavity, so also bringing the liquid containing the medicinal agent into contact therewith.

In the event that it is intended for the medicinal liquid to remain in contact with the walls of the ear cavity for a considerable time, the collar member 4 may be pulled off from the elastic stocking 15, so that it does not disturb the user, example during sleep. Complete removal of the insert will be achieved by pulling the tail 15a of the elastic stocking 15 out from the cavity.

In a further variant of the invention, shown in FIGS. 10 and 11, the operating element 2 exhibits, when in the radially compressed configuration, a substantially smooth outer surface, while when it is in the radially expanded configuration its outer surface exhibits an irregular profile with numerous projections which turn back during the expansion phase towards the first end 3a, so as to improve the effectiveness of the mechanical cleaning action of the insert without compromising the ease with which it may be introduced into the ear cavity. This advantageous feature is achieved by the provision of notches 17 directed from the surface towards the inside of the operating element, along directions inclined relative to the longitudinal axis and directed towards the free end 3b.

FIGS. 12 to 14 show a second example of embodiment of the invention, in which similar details are assigned the same numbers as in the previous example.

The insert 100 shown here differs from the insert of the previous example in that the expansion action of the operating element 2 is brought about not so much by absorption of a liquid, but by the elastic properties of the material from which it is made (for example expanded latex or expanded polyurethane or other elastomers).

The operating element 2 is held in radially compressed configuration by a containment sleeve 101 mounted telescopically on the collar member 4, the longitudinal extent of which is appropriately increased relative to the previous solution.

The containment sleeve 101 can slide axially on the collar member 4 between a first position in which it surrounds the operating element 2, holding it in the compressed configuration (FIG. 12), and a second position in which the operating element emerges from the sleeve 101 and is free to expand radially (FIG. 14).

The sleeve 101 is furthermore equipped on the opposite end from the operating element 2 with a flange 102 which permits the sleeve to be gripped manually and moved.

FIGS. 15 and 16 show a third example of an insert, designated 200, produced according to the present invention, the similar details of which are assigned the same numbers as in the previous examples.

In this case, expansion of the operating element is brought about by the particular shape thereof and by being subjected to axial thrust caused by appropriate thrust means provided in the insert 200.

The operating element 201 is held in axially sliding manner within the collar member, which takes the form of a sleeve 202, and has, in a median zone, an internal chamber 203 in which is accommodated a head 204 of a rod 205 which extends axially through the passage 5a until it emerges from the sleeve 202 at the opposite end from the operating element 201, such that it may be actuated manually. In this case, the rod 205 constitutes the thrust means.

The chamber 203 is defined laterally by the outer jacket of the operating element 201 and, on the side facing towards the free end 3b, by shoulders 206. Provision of the chamber 203 brings about a reduced thickness of the outer jacket defining it and preferential yield zones 207 are consequently defined on the outer jacket, thanks to which the free end 3b of the operating element 201 turns partially outwards when the head 204 is thrust against the shoulders 206.

In the radially compressed configuration, the operating element 201 is virtually completely inside the sleeve 202 (see FIG. 15). The axial thrust exerted manually on the rod 205 thus initially results in the emergence of the free end 3b and the preferential yield zones 207 from the sleeve 202. Subsequent axial thrust causes the free end 3b to turn outwards as a result of the torque generated by the thrust on the shoulders 206 which is not counteracted by the sleeve 202 nor by the resistance of the outer jacket at the level of the preferential yield zones 207 (see FIG. 16).

Interacting stop means are also provided to limit the axial travel of the rod 203 which respectively take the form of a shoulder 208 on the rod and tongues 209 extending from the sleeve 202.

FIGS. 17 and 18 finally show an operating element 300 of a fourth example of embodiment of the invention, in which similar details are assigned the same reference numbers as in the previous examples.

The operating element 300 differs from that of the previous example in that the thrust means comprise, instead of the rod, an expansion body 301, accommodated in the chamber 203 within the operating element 300 and made from a hydrophilic material which can expand by absorption of liquid.

As in the previous case, axial expansion of the body 301 causes the free end 3b of the operating element 300 to turn partially outwards.

A fourth example of embodiment of the invention, not shown in the appended drawings, provides for expansion of the operating element 2 by means of expansion with gas. To this end, the operating element is covered with an impermeable and deformable membrane, and, preferably, the gas is evolved in situ following a reaction of one or more chemical compounds with a liquid (e.g. water). Said compound may suitably be dispersed within the operating element, and the liquid may be introduced therein through the internal tubular duct, as in some of the previously described examples.

Examples of suitable chemical compounds are calcium bicarbonate in combination with a weak acid, such as citric acid or boric acid.

The present invention thus solves the problem described above with reference to the cited prior art, at the same time providing numerous further advantages, including the possibility of effectively removing any excess water present within the ear cavity and the possibility of making effective use of existing, commercially available liquid products for cleaning or medicating the ear cavity.

The insert according to the present invention is furthermore also ideally suited for sale in combination with vials comprising a liquid having a detergent or medicinal action and specifically sized to be engaged on the insert, and/or in combination with an adapter sleeve to permit the use of existing commercially available containers.

1.-26. (canceled)

27. An insert for treating a human or animal body cavity, comprising an operating element located at one end of said insert intended to be introduced into said cavity and to come into contact with internal walls of said cavity, wherein said operating element is expandable from a radially compressed configuration, in which said operating element may be introduced into said cavity without substantial interference with said internal walls, to a radially expanded configuration having a substantially larger cross-section relative to said radially compressed configuration, in which radially expanded configuration said operating element is pressed against said internal walls of said cavity.

28. The insert according to claim 27, wherein said operating element is supported and held at one first end thereof by a rigid collar member surrounding said first end of the operating element.

29. The insert according to claim 28, wherein a containment sleeve is mounted telescopically on said collar member in an axially sliding manner between a first position in which it surrounds said operating element, holding it in said radially compressed configuration, and a second position in which it is at least in part pulled back from said operating element, permitting the operating element to change into said radially expanded configuration, in which a free end opposite to said first end is partially turned outwards towards said first end.

30. The insert according to claim 29, wherein said operating element comprises an elastic material.

31. The insert according to claim 28, wherein said operating element comprises a hydrophilic material which is expandable by absorption of a liquid and an internal tubular duct is provided which is located axially in said insert to transfer a liquid from outside said cavity towards said operating element.

32. The insert according to claim 31, wherein there is located on said collar member a passage to place said internal tubular duct in fluid communication with the outside of said insert.

33. The insert according to claim 32, in which there is engaged on said collar member, from the axially opposite side from the said operating element, a container containing a liquid to be introduced into said cavity.

34. The insert according to claim 33, wherein said liquid comprises an agent having a detergent action or an agent having a medicinal action.

35. The insert according to claim 33, wherein said container comprises a mouth engaged on said collar member and on said mouth there are located internal radial tongues capable of holding said container in engagement on the collar member, said tongues defining respective circumferential openings permitting the passage of liquid between the collar member and the container, externally to said operating element.

36. The insert according to claim 35, wherein said human or animal body cavity comprises an ear cavity, and wherein said mouth comprises an annular projection which extends towards the outside of said mouth and is capable of interfering with the outer ear in order to limit the distance said insert is introduced into said ear cavity.

37. The insert according to claim 32, in which there is engaged on said collar member, from the axially opposite side relative to said operating element, an internally hollow adapter sleeve to permit connection with a dispensing spout of an external liquid container.

38. The insert according to claim 37, wherein said adapter sleeve is engaged on the outside of said collar member and comprises radial tongues capable of holding the adapter sleeve in engagement on the collar member, said tongues defining respective circumferential openings to permit the passage of liquid between the collar member and the adapter sleeve, externally to said operating element.

39. The insert according to claim 37, wherein said human or animal body cavity comprises an ear cavity, and wherein said adapter sleeve comprises an annular projection which extends towards the outside of said adapter sleeve and is capable of interfering with an outer ear in order to limit the distance said insert is introduced into said ear cavity.

40. The insert according to claim 28, in which the operating element in said radially compressed configuration has a substantially cylindrical shape and in said radially expanded configuration has a bell-like shape, in which one free end opposite to said first end is partially turned outwards towards said first end.

41. The insert according to claim 27, in which said operating element comprises attachments which extend radially inwards and are configured in such a manner as to press one against the other during the expansion phase, so as to promote radial expansion of said operating element.

42. The insert according to claim 28, in which, on one free end, opposite to said first end, there are provided a plurality of concentric crowns arranged in close succession one after the other along a longitudinal axis (X) of said operating element, said crowns, in said radially compressed configuration, extending parallel to the axis X and being coaxially closed one around the other, and, in said radially expanded configuration, extending parallel to a plane substantially perpendicular to said longitudinal axis (X).

43. The insert according to claim 28, in which said operating element exhibits a substantially smooth outer surface in said radially compressed configuration, while in said radially expanded configuration it exhibits an outer surface with an irregular profile on which are defined a plurality of projections which are partially turned outwards towards said first end.

44. The insert according claim 28, wherein said operating element is covered by an elastic stocking.

45. The insert according to claim 44, wherein said collar member is removable from said operating element and said elastic stocking is provided with a tail so as to facilitate subsequent removal of said insert from said cavity.

46. The insert according to claim 28, in which, at a level of a median zone of an outer jacket of the operating element, there are located preferential yield zones, such that a free end of the operating element opposite to said first end is partially turned outwards towards said first end as a result of axial thrust exerted by thrust means provided on said operating element.

47. The insert according to claim 46, in which said operating element can slide axially within said collar member between a first position, in which the operating element is retracted inside the collar member in radially compressed configuration, and a second position, in which the operating element projects from said collar member and is in radially expanded configuration, said operating element being caused to slide from said first to said second position by said thrust means.

48. The insert according to claim 47, in which said thrust means comprise a rod which is inserted slidingly in said collar member and abuts against said operating element, said rod projecting from the opposite side of the operating element in order to be actuated manually.

49. The insert according to claim 46, in which said thrust means comprise an expansion body made from a hydrophilic material which is axially expandable by absorption of a liquid, said expansion body being accommodated within said operating element.

50. The insert according to claim 27, in which said operating element is covered with an impermeable and deformable membrane, and within said operating element there is dispersed at least one compound capable of releasing a gas as the result of a chemical reaction in the presence of a liquid introduced through a tubular duct located within said operating element.

51. A kit for cleaning a human or animal body cavity, comprising an insert according to claim 27.

52. The kit according to claim 51, further comprising an adapter sleeve which may be engaged at a first end onto said insert and at the opposite end onto a dispensing spout of a liquid container.

53. The kit according to claim 51, further comprising a dispensing container for a detergent or medicinal liquid.

54. The kit according to claim 51, wherein the human or animal body cavity comprise an ear cavity.
